(19) United States
(12) Patent Application Publication
(10) Pub. No.: US 2007/0023540 A1
(43) Pub. Date: Feb. 1, 2007

(54) Fragrance Delivery for Multimedia Systems

(76) Inventor: Raymond K. Selander, Hopewell Junction, NY (US)

Correspondence Address: INTERNATIONAL FLAVORS & FRAGRANCES INC., 521 WEST 57TH ST, NEW YORK, NY, 10019, US

(21) Appl. No.: 11/532,677

(22) Filed: Sep. 18, 2006

Related U.S. Application Data
(63) Continuation-in-part of application No. 11/069,097, filed on Mar. 1, 2005.
Application No. 11/069,097 is a continuation-in-part of application No. 10/950,903, filed on Sep. 27, 2004.
Application No. 10/950,903 is a continuation-in-part of application No. 10/791,923, filed on Mar. 3, 2004, now abandoned.

(51) Int. Cl. A61L 9/00; A24F 25/00; A62B 7/08

(52) U.S. Cl. 239/34; 422/5; 422/124

(57) ABSTRACT

Apparatus and methods for fragrance sensory stimulation connected to a multimedia source that has at least one audiovisual signal connected to an audiovisual display, and also has fragrance information synchronized with the audiovisual signal is disclosed. In preferred embodiments, a fragrance generator processes the fragrance information into a fragrance signal and at least one fragrance control system accepts a fragrance signal as an input, and generates a control signal and a controlled airflow. In turn, a fragrance delivery system preferably comprising a plurality of fragrance chemicals in communication with the controlled airflow, selectively volatilizes the fragrance chemicals is so that a short burst of fragrance is sensed by the subject. In certain embodiments, the fragrance delivery system comprises an indexing turret containing a plurality of ports, each port containing one of said plurality of fragrance chemicals. In other preferred embodiment, a multiport micro-valve or array of such valves contains an absorbent material impregnated with a fragrance in each port.

### STATUS OF RELATED APPLICATIONS

This application is a continuation-in-part of U.S. Ser. No. 11/069,097 filed Mar. 1, 2005, which is a continuation-in-part of U.S. Ser. No. 10/950,903 filed on Sep. 27, 2004, which is a continuation-in-part of U.S. Ser. No. 10/791,923 filed on Mar. 3, 2004, the contents of all of which are all hereby incorporated by reference as if set forth in their entirety.

### FIELD OF THE INVENTION

The present invention relates to fragrance delivery systems, and more particularly to fragrance delivery systems that are synchronized with audio and/or visual presentations to add fragrance to a display of sights and/or sounds.

### BACKGROUND OF THE INVENTION

The “experience” of audiovisual entertainment has progressed from silent films and monaural recordings to today's visually stunning digital images and advances in sound reproduction such as the “surround sound” found in both commercial theatres and homes. Moreover, current computer-based games and games played on gaming platform employ the same advanced graphics and sound qualities found in film or video. These technologies create an audiovisual “experience” that immerses the user in a film or game like never before, stimulating sight, sound and even tactile sensations through deep bass vibrations provided by subwoofers and tactile feedback provided by some game controllers. There remains, however, one form of sensory perception that is not stimulated—the sense of smell.

Numerous prior art systems have attempted to provide a scent to the environment that compliments or correlates to an audiovisual stimulus. Examples would be the smell of burning rubber coordinating with the screech of tires, or the scent of flowers when a corresponding image appears. Prior art systems have primarily been directed to introducing fragrances to large environments, such as an entire theater. These systems have by and large suffered from the problem that a fragrance will linger long after the coordinating audiovisual input has changed, and may in fact be difficult to replace with another scent as the scenes change, the typical result being a mixture of several fragrances that become an indistinct muddle.

None of these prior art devices, however, provides a useful and commercially viable system for fragrance delivery to enhance an audiovisual presentation. Therefore, there remains a long-felt yet unmet need for providing it would therefore be desirable to provide. It would further be desirable to provide such improvements in a manner that permitted their application across a variety of situations and that permitted their implementation in a cost-effective manner.

### SUMMARY OF THE INVENTION

Accordingly, it has now been found that these and other shortcomings of the prior art can be overcome by providing an apparatus for fragrance sensory stimulation that is connected to a multimedia source that has at least one audiovisual signal connected to an audiovisual display, and also has fragrance information synchronized with the audiovisual signal. In preferred embodiments of the present invention, a fragrance generator processes the fragrance information into a fragrance signal and at least one fragrance control system accepts a fragrance signal as an input, and generates a control signal and a controlled airflow. In turn, a fragrance delivery system preferably comprising a plurality of fragrance chemicals in communication with the controlled airflow, selectively volatilizes the fragrance chemicals is so that a short burst of fragrance is sensed by the subject. In certain embodiments, the fragrance delivery system comprises an indexing turret containing a plurality of ports, each port containing one of said plurality of fragrance chemicals. In other preferred embodiment, a multiport micro-valve or array of such valves contains an absorbent material impregnated with a fragrance in each port.

In certain embodiments, the system is constructed as two subassemblies, in which a transmitter is connected to the fragrance generator and a receiver is connected to the fragrance creation system, and the transmitter sends the fragrance signals to the receiver to control the creation of synchronized bursts of fragrance, most preferably provided by a compressor regulated by a microprocessor and including a high-pressure cutoff sensor so that a short burst of pressurized air having a duration of less than five seconds is created. In certain preferred embodiments of the apparatus of the present invention, the fragrance control system is mounted on a user's body, while in other preferred embodiments, the fragrance control system is mounted on a chair. Similarly, in certain preferred embodiments of the apparatus of the present invention, the fragrance delivery system is mounted on a user's body, while in other preferred embodiments, the fragrance control system is mounted on a chair. In other embodiments, a user interface is mounted to the user via an apparatus such as a mask, helmet, headset or earpiece. Alternatively, the interface can be placed adjacent the user, e.g., on a desk, or incorporated into another device such as a steering wheel controller for a game system.

The present invention thus provides a fragrance producing system synchronized to an audiovisual medium that preferably includes a fragrance delivery system for providing a short burst of air containing one of a plurality of fragrance chemicals to an individual user coordinated by receiving and processing a signal contained within the audiovisual medium that has been transmitted to a fragrance creation system in which the signal activates a compressor that selectively volatilizes the fragrance chemical, wherein the plurality of fragrance chemicals are disposed within an apparatus that selectively mixes one of the plurality with a burst of pressurized air generated by said compressor. Most preferably, the signal contained within the audiovisual medium is transmitted via and RF transmitter to an RF receiver connected to the fragrance creation system, and the apparatus that selectively mixes one of the plurality with a burst of pressurized air generated by the compressor comprises a turret with a plurality of ports, and each of the ports includes an absorbent material and a fragrance chemical. Alternatively, the apparatus that selectively mixes one of the plurality with a burst of pressurized air generated by the compressor comprises an array of micro-valves, and each of the micro-valves includes an absorbent material and a fragrance chemical.

Finally, the present invention also discloses methods of synchronizing a fragrance stimulus to a user with one or more audio and visual stimuli by providing a fragrance track to an audiovisual medium to provide a coded signal that correlates to a type of fragrance desired to be released at a pre-determined time, and then determining the divisions within the medium in which fragrance is to be delivered and creating a sequence of fragrances. A device having the requisite number of ports containing a sufficient variety and quantity of fragrance to correspond to the sequence of fragrances is provided and loaded with the fragrance. Next, a signal is processed to activate a compressor and connect the device with an airflow that the correct fragrance chemical is volatilized when a burst of air reaches the fragrance chemical. In certain embodiments. the step of providing a fragrance track comprises programmatic methods direct the system to generate a scent for a pre-determined brief period of time.

### BRIEF DESCRIPTION OF THE DRAWINGS

FIG. 1 is a block diagram illustrating a preferred embodiment of the present invention;

FIG. 2 is a partially schematic, partially perspective view of a preferred embodiment of a fragrance creation system used in the system shown in FIG. 1;

FIG. 3 is a partially schematic, partially perspective view of a preferred embodiment of the delivery creation system used in the system shown in FIG. 1;

FIG. 4 is a front elevation view of a preferred embodiment of a user interface in the form of a mask;

FIG. 5 is a perspective view of a preferred embodiment of a user interface in the form of a “desktop” device;

FIG. 6 is a side elevation view of a preferred embodiment of a user interface in the form of a head-mounted device;

FIG. 7 is a side elevation view of a preferred embodiment of a user interface in the form of an ear-mounted device;

FIG. 8 is a side elevation view of a preferred embodiment of a user interface in the form of a user-worn pendant;

FIG. 9 is a perspective view of a preferred embodiment of a user interface in the form of a helmet;

FIG. 10 is a perspective view of a preferred embodiment of a user interface in the form of a steering wheel gaming controller;

FIG. 11 is a perspective view of an array of micro-valves used to dispose of fragrance.

FIG. 12 is a block diagram of the operation of a fragrance dispersal system utilized in conjunction with preferred embodiments of the present invention;

FIG. 13 is partially schematic illustration of the components used in a microfluidic spray device; and

FIG. 14 is an elevation view of a micropump and capillary needle used in the device illustrated in FIG. 13.

### DETAILED DESCRIPTION OF THE PREFERRED EMBODIMENTS

The implementation of the present invention is in several preferred embodiments, discussed below, along with several illustrative examples. The embodiments of the invention described below are provided for the purpose of understanding the invention and are not meant to be limiting.

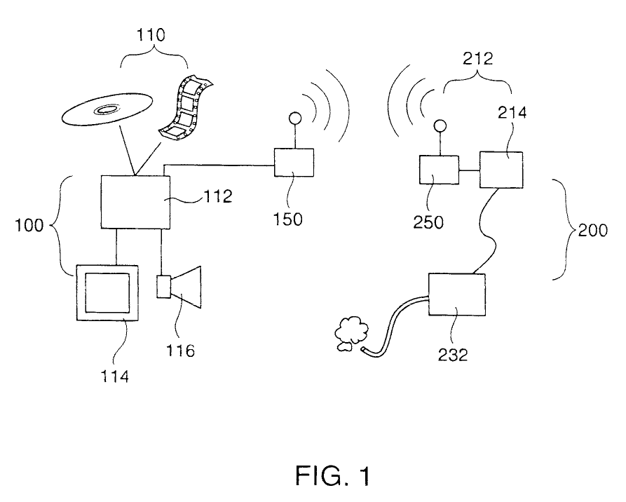

Referring now to FIG. 1, a first embodiment of a system employing the present invention is illustrated. There are two main sub-systems that make up this embodiment, namely, the fragrance generator 100 and the fragrance delivery system 200, each of which is discussed in further detail below. In the preferred embodiment illustrated in FIG. 1, the fragrance generator 100 and the delivery system 200 are preferably connected by a wireless system comprising a transmitter 150 and a receiver 250, which are most preferably RF devices, while in other embodiments, such devices and subassemblies can be connected by wires, or integrated into a single unit.

The fragrance generator 100 is comprised of a medium 110 and a media player 112. As known in the art, the medium 110 and media player 112 can be any one of a number of systems, either digital, analog or some combination of such systems, that contains information and converts the information into a signal that can be use for display. Thus, the medium 110 will comprise one or more “tracks” such as a video track, a sound track and, in accordance with the present invention, a fragrance track. The media player 112 will read or process the medium 100 and create signals that can be displayed by devices such as a visual display 114 and an audio display 116. However, in accordance with the present invention, the scent track described above is transmitted to and processed by a fragrance control system 212 that may or may not be integral with the media player 112. As discussed in further detail below and well known in the art, there are a number of systems that contain a scent “medium” (not illustrated in FIG. 1) that can be “played” by the fragrance control system 212 to control the selective release one or more scents in coordination with the information being provided to the visual display 114 and audio display 116. The coordination of emitting various scents during playback of a video, or during the playing of a video game is disclosed in U.S. Pat. No. 6,654,664-Manne and in U.S. Patent Application Publications 2001/0008611 and 2002/0036358, none of which are admitted to be prior art to the present invention.

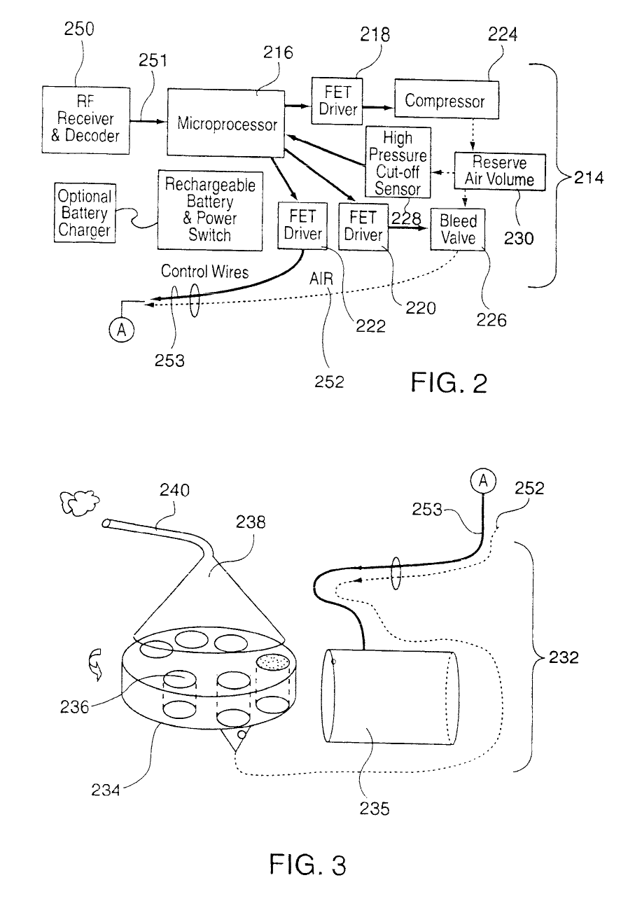

As illustrated in FIG. 1, in certain embodiments of the present invention, the fragrance creation system 212 is divided into two sections, although they may be physically integrated if desired. In accordance with one aspect of the present invention, the fragrance creation system 212 provides short bursts of synchronized fragrances coordinated with audio or visual information on a real-time basis. A first section of the frequency creation system 212 is the fragrance control system 214. Referring now to FIG. 2, a block diagram of one preferred embodiment of a fragrance control system 214 is illustrated. The fragrance control system 214 can be either connected directly to the fragrance generator 100, or as described above, may connected via an RF transmitter 150 and receiver 250 as described above with reference to FIG. 1. In such an embodiment, the RF receiver 250 will include a decoder for RF signal drivers. In any event, a fragrance signal 251 is provided to a microprocessor controller 216, which in turn controls a set of FET drivers 218,220,222 that control, respectively, a compressor 224, a bleed valve 226 and other portions of the device. An additional high-pressure cutoff sensor 228 monitors a reserve air volume 230 and the bleed valve 226. In operation, the microprocessor controller 216 activates the compressor 224. When the upper limit of pressure is achieved, the high pressure cutoff sensor 228 signals the microprocessor to deactivate the compressor, thus creating and maintaining a pressurized system so that when appropriate a short burst of pressurized air is available to be conducted elsewhere in the system. This system may be either be battery powered or wired to a current source. In operation, the microprocessor converts the fragrance signal 251 into a signal that controls a regulated flow of air 252 created by the compressor and a control signal 253, both of which are carried to a fragrance delivery system 232, discussed in detail below with relation to FIG. 3. Thus, the present invention provides precise control of an air pressure reserve that assures immediate and accurately metered delivery of a bolus of scented air that is synchronized with the game, movie or other information, and further that can be provided either as a mixture or a sequential series of scents.

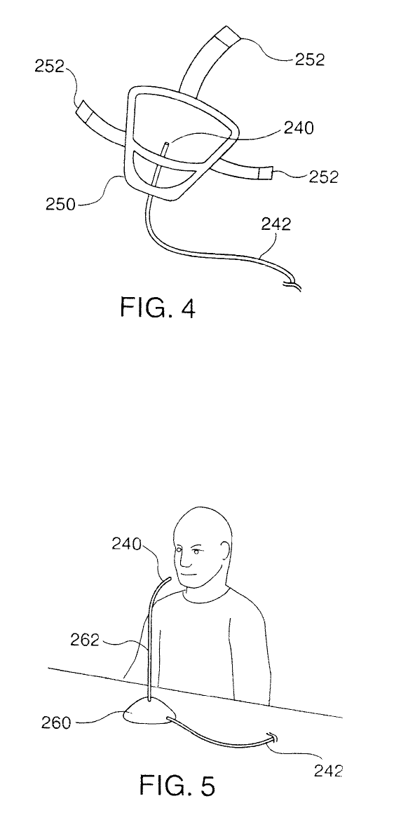

As will be appreciated by those of skill in the art, the system described with relation to FIG. 2 is relatively easy to construct and will be a relatively small-scale unit, thereby permitting devices made in accordance with this aspect of the present invention to be integrated into a compact unit at a relatively low cost to enable economic mass production and widespread implementation. Moreover, such a construction will permit the frequency control system 214 to be constructed in embodiments that can be body-worn, e.g., on a waist belt, or conveniently and unobtrusively mounted to a theatre chair, airline or vehicle passenger seat, or a seat in the home. Further details of the interface to the end user are described and illustrated below with reference to FIGS. 4-10.

Referring now to FIG. 3, a partially schematic, partially perspective view of a preferred embodiment of a fragrance delivery system 232 is illustrated. As discussed above with reference to FIG. 2, a controlled flow of air 252 and an electrical signal 253 are carried from the fragrance control system 214. The controlled flow of air 252 is connected via a conduit such that it flows into and through a turret 234 that houses a plurality of fragrance chemicals 236 that are volatilized when the controlled flow of air 252 enters the turret 234. In the preferred embodiment illustrated, the turret 234 includes a plurality of ports or wells 236 that contain an absorbent material that is impregnated or “charged” with a particular scent. The electrical signal 253 controls a solenoid 235 that indexes the turret to a position that corresponds to a particular port 236. Depending upon the use, e.g., commercial or home use, the ports 236 may be accessible so they can be recharged or reloaded with fragrance chemicals, or the entire turret 234 may be a disposable structure that is replaced each time the system is used, or replaced after a specified number of viewings of a film or sessions of game play. In certain other preferred embodiments, the turret 236 can be replaced by an array of micro-valves, each disposed over a port 236 containing fragrance chemical, and the fragrance is volatilized upon opening of the valve by the signal 235 and the passage of the airflow 252 through the open orifice of the valve. As illustrated in FIG. 11, one embodiment of an array of micro-valves 334 includes a valve block 335 that has a series of wells 336 and that is covered by a header 338 (shown in an exploded view) that has a corresponding check valve 339, such as, for one example ⅛ inch (0.125″) ID valves P/N 98553-10 available from Cole Parmer. As explained above, each check valve 339 will selectively receive a blast of gas and volatilized fragrance that are then mixed and or sequentially dispersed through the valve block 334 and into the conduit 242 that connects to the nosepiece 240 (not illustrated in FIG. 11).

Still referring to FIG. 3, as the airflow passes through the turret 236, it is collected by a manifold hood 238, which in turn conducts the airflow, which now carries the selected scent, to a nosepiece 240 or similar structure that directs the fragrance to the nostrils of a single user. The use of a nosepiece and related apparatus to conduct a flow of scented air to a wearer is known in the art, for example, U.S. Patent Application Publication 2004/0003812, which is not admitted to be prior art to the present invention, discloses a portable fragrance control device that releases scent directly into a user's nose via diffusion. The device disclosed is structurally similar to the headsets commonly used for listening and/or speaking. The fragrance delivery system 232 is thus controlled by the signal provided by the fragrance control system 214 and “powered” by the compressor 224 that forms part of that same subsystem. Preferably, the fragrance delivery system 232 is constructed to be lightweight and can be either worn on the body or integrated into the headrest of a chair, but in either case it is preferred that the nosepiece 240 be disposed proximate the wearer's or user's nose so that the fragrance is conducted directly to the individual and does not permeate the environment generally. Details of devices for mounting or placing the nosepiece proximate an end user are described and illustrated below with reference to FIGS. 4-10. Moreover, as mentioned above, it is further desirable that the fragrance delivery system 232 emits short bursts of fragrance at the appropriate synchronized time to enhance the experience of a game, movie or similar audiovisual presentation.

Thus, in operation, those of skill in the art will appreciate that a fundamental requirement of the present invention is that the medium contain an additional “track” that is designed to provide a coded signal that correlates to the type of scent desired to be released at the particular temporal point in the audio or visual presentation. Alternatively, programmatic methods could be employed, e.g., a short algorithm that directs the system to generate a scent upon intervals, e.g., generating short bursts (for example, less than three seconds) of pine scent every thirty seconds during prolonged scenes in an evergreen forest. Such signals, whether part of a “track” or programmed are easily integrated into various forms of media, such as digital discs (CDs, DVDs, etc.), game cartridges, the magnetic tracks found on commercial theatre film, videotape and audiotape, and various other forms of magnetic media. Typically, the numbers of scenes or similar divisions within the medium in which fragrance will make an impact or otherwise be appropriate are identified and a sequence of fragrances is created. A turret or array of valves having the requisite number of ports containing a sufficient variety and quantity of fragrance to correspond to the sequence of fragrances is designed and provided. In operation, the system is loaded with the correct turret or valve array and the user is positioned so the nosepiece described above will transmit fragrance to the wearer in a synchronized fashion. The “fragrance track” provides a signal that is processed to activate the compressor and indexes the turret so that the correct fragrance chemical is volatilized when the burst of air reaches the fragrance chemical.

In preferred embodiments, a micropump dispersal system will operate dependably in the context of the present invention without creating ozone or other undesirable atmospheric byproducts or “fallout,” yet delivering an even volumetric distribution of a fluid (i.e., a fine dispersal of fragrance) over the life of the device, or at a minimum until the volume of liquid in the reservoir is exhausted. In preferred embodiments of the present invention, a microfluidic spray device is incorporated and computer controlled to provide a fine spray of fragrance chemicals.

Referring to FIG. 12 a block diagram of the microfluidic dispersal system 400 integrated into certain preferred embodiments of the present invention is illustrated. An activation device 410 creates a signal that activates dispersal of one or more scents. As explained above, the activation device is typically but not necessarily an electronic signal carried by an audiovisual soundtrack. In alternate embodiments, the activation device can be a timer or a clock, or a motion sensor or photocell. In any embodiment, a stimulus of some kind generates a signal, which in turn activates a microprocessor or an ASIC (Application Specific Integrated Circuits) 420, explained in further detail below, which operates the dispersal system. The integrated circuit in turn controls the operation of a micropump 430, which is preferably a piezoelectric device. The micropump 430 is connected to at least one source of fragrance chemical 440, but most preferably is connected to an array of fragrance chemicals, as explained below. The micropump 430 pressurizes the fragrance chemical 440 and the resulting atomized or volatilized fragrance is delivered to the environment or to the user, as explained with reference to other embodiments of the present invention. In preferred embodiments, the micropump 430 is a piezo electric device that provides constant volumetric flow over the life of the fluid supply in the fragrance chemical reservoir 440. Typically, electronic controls built into the microprocessor 420 provide precise voltage and frequency (in certain preferred embodiments the optimal frequency is 80 Hz) to the micropump 430 to deliver consistent volumetric delivery. Thus, as known in the art, the microprocessor 420 may provide for customized control of the operations of the micropump 430 and associated components, as well as providing options, such as on/off cycling, photocell operation, burst mode operation, motion activation and the like.

The microfluidic spray device is illustrated in FIG. 13. As shown, in preferred embodiments, multiple sources of fragrance chemicals 440,442,444 are provided and are connected to the pump 430 in a sequence determined by the microprocessor 420. In certain embodiments, the sequence will follow directly from the activation signal. For example, if there is a scene in a film that includes a vehicle crash, the smells of engine exhaust, burning rubber, gasoline and smoke might all sequentially follow from a single activation signal in the audiovisual source. In alternate embodiments, an activation signal might initiate a longer sequence of minutes or hours where a variety of scenes are dispersed in a sequence to tell a “story” via the scents themselves. In any embodiment, it is preferred that the micropump 430 connect vial a capillary tube 434 to a charged needle dispersal valve 452, which is described in greater detail below. The charged needle system provides an output of finely divided droplets of scent chemical that disperse evenly and volatilize the scent efficiently without resort to carrier gases or high pressure. Although the system described will volatilize a liquid into a fine dispersal, it is preferable in certain embodiments to add a fan 460 to accelerate and assist in the delivery of fine fragrance to fill a room.

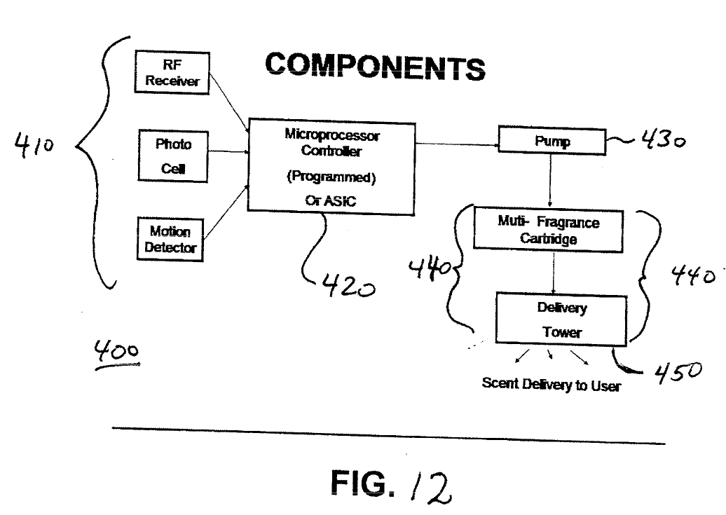

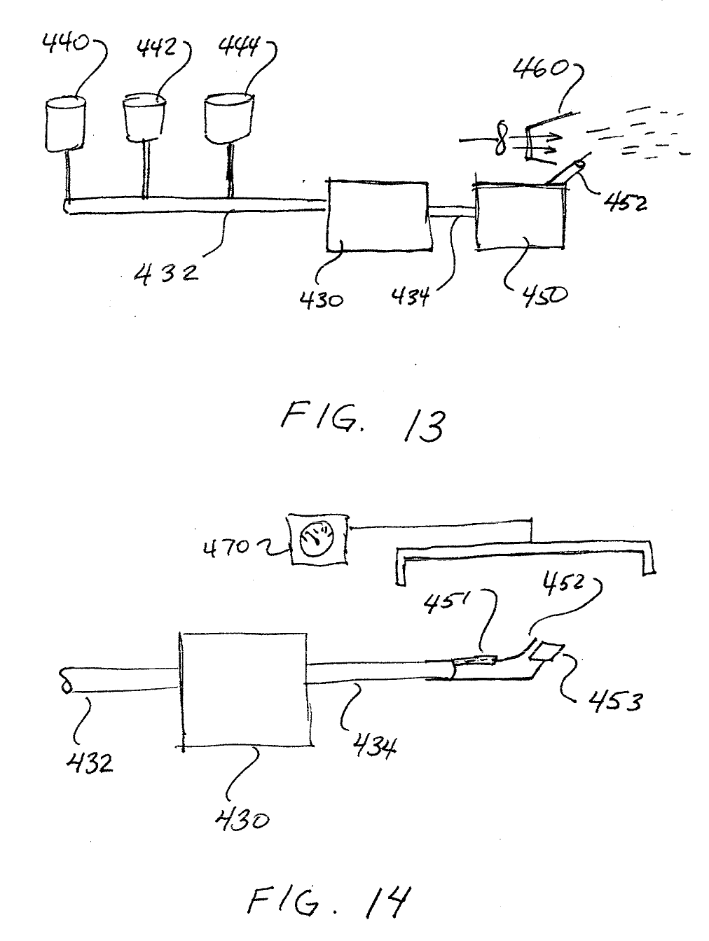

Further details of the microfluidic spray device are shown in FIG. 14. A tube 432 carries fragrance chemical into the pump 430 and, as mentioned above, a plastic capillary (tube) 434 of about 160 microns I.D. with a wall thickness of about 19 microns exits the pump 430. Inside this plastic capillary 434 is an internal (exposed to passing fluid) stainless steel metallic wire (conductor) 451 of about 19 microns O.D that terminates at its distal end as a dispersal needle 452. A positive direct current, preferably created by 2500 volts DC or less, is applied to nebulize fine fragrance oils as well as other fluids (e.g. sanitizers such as tetraethylene glycol (TEG)) without the addition of carrier gases, which have the potential of creating ozone or other harmful byproducts. The system uses a metallic ground foil 453 to provide a target plane that attracts the positively charged nebulized fluid. In other embodiments, the ground foil is positively charged if the needle is negatively charged. This target plane 453 is preferably is maintained at a fixed distance between 0.25 inches and 0.5 inches from the exit port of the needle 452. The needle 452 is insulated from all other components such as the fragrance chemical reservoir 440 and micropump 430. Although the length of the needle 452 is not critical to the operation, in preferred embodiments it is approximately 0.25 inches or greater in length for ease of manufacturing. To fine-tune the system, it is understood by those skilled in the art that the capillary length is part of the fluidic resistance. The needle 452 is readily made longer or shorter in correspondence with the diameter of the capillary 434 to reach the desired spray volume output. Preferably, the exit port of the capillary 434 and the end of the needle 452 have a blunt end (perpendicularly cut) that optimizes the nebulization process. The internal wire (conductor), inside the capillary, ends at the blunt end of the capillary. Materials that are compatible to fine fragrance oils are used in the implementation of our prototypes. They include Dupont Kalrez®, silicone and Polyphenylsulfone (PPSU). Materials that are avoided include Polycarbonate and Polystyrene, which are poor for handling fine fragrance oils.

Although calibration of the system described herein is readily accomplished with conventional available equipment is time consuming and not very accurate, it does not require undue experimentation. However, each fragrance oil used presents another set of parameters (viscosity, conductivity, surface tension, etc) all of which affect the performance of the system and require re-calibration. Therefore, in certain preferred embodiments, the system will include a microprocessor, photodiode array, and light source disposed in the vicinity of the needle 452. The calibration system 470 is shown graphically in FIG. 14. Using discrete electronic components to make a calibration system that is dependable and accurate and provides real time measurements to permit more efficient and precise calibration. In these embodiments, the microprocessor control preferably includes a circuit design that permits the dispersal system describe herein to perform reliably and consistently. Those embodiments using ASIC's (Application Specific Integrated Circuits) will permit miniaturization of the device allowing for battery-powered embodiments. The newly available Chip MAX668 along with a Microprocessor (PIC 18F 1220) and new software algorithms programmed into the PIC provide an optional Sine Wave or Square wave to drive the Micropump (Piezo) through Pulse Wave Modulation (PWM).

An advantage of the electrostatic systems described above is that allergens and the like are destroyed when in the discharge of the device. When high voltage is applied to fragrance oils, which include for example TEG (triethylene glycol) an attraction to airborne particles results causing them to precipitate. Thus one mechanism of action is that if the fragrance contains TEG or similar compounds, bacteria would become attached to these molecules and be destroyed as the oils are nebulized by electrostatic action. In addition to bacteria, other organic (or possibly even inorganic) particulates such as tobacco smoke, dander and the like can be beneficially removed from the airflow by the electrostatic nebulization process described above.

Referring now to FIGS. 4-8 additional preferred embodiments of apparatus that permit use of the present invention by an end user are shown. As discussed above, in certain embodiments, a nosepiece 240 is provided so that a fragrance is transmitted to an end user. As shown in FIG. 4, in one preferred embodiment, the nosepiece 240 is worn by making it part of a mask 250 that is worn over the front section of the face of a user. A nosepiece conduit 242 conducts both fragrances via one or more conduit tubes, and in some embodiments electrical signals to the nosepiece 240. The mask 250 is preferably provided with head straps 252 so that the mask 250 will remain firmly affixed while the user turns or moves. This embodiment will be particularly adaptable for use with video or computer games involving sports or other action simulations in which the user is immersed in a virtual or near-virtual experience. As know in the art, a face-worn mask can be comprised of a single molded part or make from a more rigid material that is padded with a softer material, e.g., a metal skeleton covered with polyvinyl or EVA foam.

Referring now to FIG. 5, an alternative embodiment of a device for placing a nosepiece 240 proximate an end user is illustrated. As discussed above with reference to FIG. 4, the nosepiece also comprises a nosepiece conduit 242. In the embodiment illustrated in FIG. 5, the nosepiece 240 is mounted to a stand 260 that is in turn placed on a suitable surface, e.g. a desktop or tabletop. A semi-rigid or bendable stalk 262 extends from the stand 260 and enables the distal end of the nosepiece 240 to be placed near the nose of the end user, as illustrated. Those skilled in the art will understand that the stalk 262 can a separate structure or can simply be a continuation of the conduit 242 if that portion of the device is formed of a material that can be bent and hold a shape, such as molded plastic tubing that is molded around a flexible metal substrate or that has wire embedded within. Alternatively, in certain alternate embodiments it may be desirable for the stalk 262 to be completely rigid.

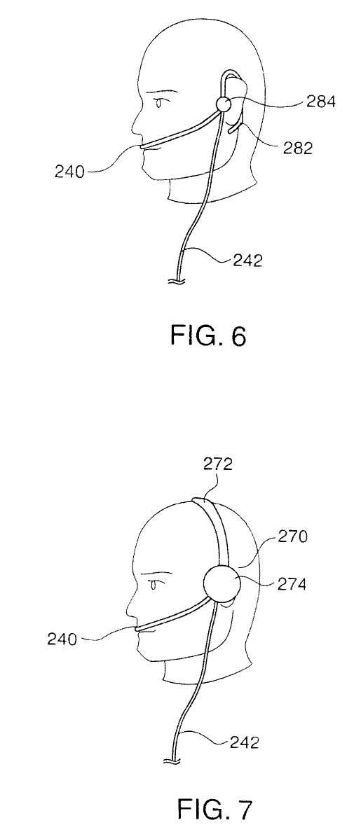

An additional embodiment of a device for placing the nosepiece 240 near a user's nostrils is shown in FIG. 6. As illustrated the end user simple wears an earpiece 282 over an ear, and from this structure, the nosepiece 240 extends. This embodiment can further include an earphone 284 attached to the earpiece 282, which will provide additional stability as the user's head moves, and can optionally integrate an earphone to reproduce an audio track. As in the other embodiments discussed herein, the device further includes a conduit 242.

Another embodiment of a device for placing the nosepiece 240 near a user's nostrils is shown in FIG. 7. In this embodiment, the nosepiece 240 extends from a headset 270 that preferably and typically includes a head strap 272. The headset may or may not include one or more headphone speakers 274. In other words, in certain embodiments, a headset structure can be employed without providing sound transmission via the structure surrounding one or both of the ears. As mentioned above with reference to FIG. 4, this embodiment is particularly well suited for action and sports games where the user might tend to undergo a greater degree of head motion than typical in more passive activity such as viewing a movie.

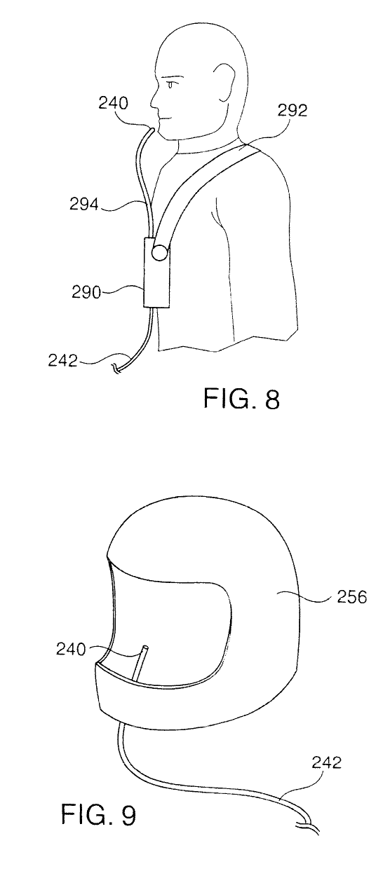

As seen in FIG. 8, another device that permits a user to “wear” the nosepiece 240 is comprised of a pendant 290 that is worn close to the body and preferably attached to the user by a neck strap 292. In this embodiment, the nosepiece 240 again extends from a stalk 294 in the manner discussed above with reference to FIG. 5 only in this embodiment rather than resting on a desk or table, the nosepiece is simply affixed to a pendant 290 that rests against he user's body. The pendant 290 provides an interface with the conduit 244, and could optionally contain additional functions, such as headphone jacks, and in such an embodiment the conduit 242 would further include wires carrying an audio signal.

Referring now to FIG. 9, a user interface in the form of a helmet is illustrated. In this embodiment, a helmet 256 is provided, as is typically known for use with gaming consoles, e.g., to add to the experience of racing games. As is also known, helmets are often employed in “virtual reality” simulators. The helmet 256 incorporates a nosepiece 240. In the embodiment illustrated, the lower edge of the helmet 256 provides a convenient mounting point for the nosepiece 240. In other helmets lacking this lower structure, the nosepiece 240 would be mounted to the helmet and extend along the side of the jawline, as illustrated in FIGS. 6-7. The conduit 242 can, as discussed above, carry both fragrance and audio or other signals, for example if earphones are built into the helmet 256. As discussed above with reference to FIG. 4, and embodiment such as the one illustrated in FIG. 9 is particularly adapted to implementation where the user will undergo significant head movement during game play or other audiovisual experience.

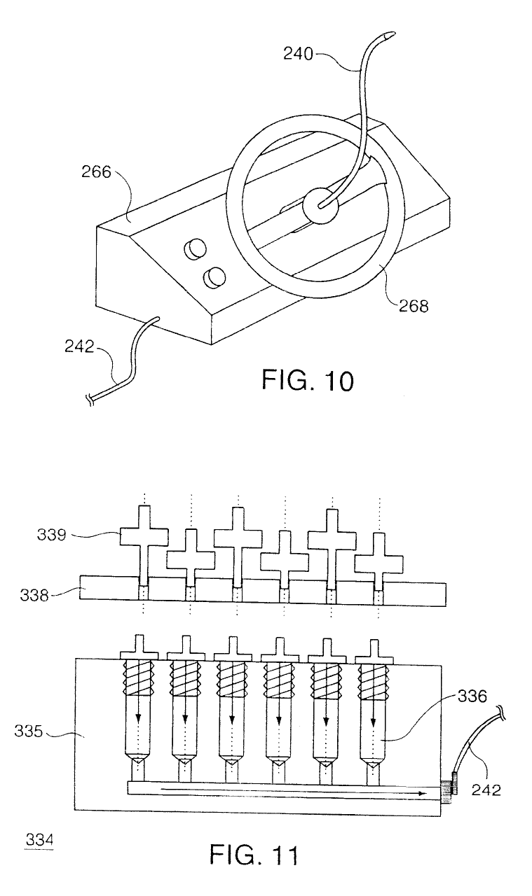

Finally, as seen in FIG. 10, a gaming system controller 266 that includes a steering wheel 268 can be adapted for use with the present invention. In this embodiment, as discussed above with reference to FIG. 5, the device is placed on a desk or table near the user and a nosepiece 240 extends upwardly toward the user. As noted above, it is particularly preferable that the nosepiece 240 be constructed so that it can be bent and retain a shape so it can be located in an appropriate position. The conduit 242 carries fragrance to the nosepiece 240 and may optionally include signal wires for game control and/or a power cord.

Upon review of the foregoing, numerous adaptations, modifications, and alterations will occur to the reviewer. These will all be, however, within the spirit of the present invention. Accordingly, reference should be made to the appended claims in order to ascertain the true scope of the present invention.

What is claimed is:

1. An apparatus for fragrance sensory stimulation comprising:
a multimedia source comprising at least one audiovisual signal connected to an audiovisual display, and fragrance information synchronized with the audiovisual signal;
a fragrance generator for processing said fragrance information into a fragrance signal;
at least one fragrance control system that accepts a fragrance signal as an input, and generates a control signal and a controlled airflow; and
a fragrance delivery system comprising a plurality of fragrance chemicals volatilized by a microfluidic dispersal system in communication with the controlled airflow, whereby at least one of said fragrance chemicals is volatilized and the volatilized fragrance is collected and sensed by a subject.

2. The apparatus for fragrance sensory stimulation according to claim 1, wherein the fragrance delivery system further comprises a capillary tube and a dispersal needle.

3. The apparatus for fragrance sensory stimulation according to claim 2, wherein the dispersal needle is electrically charged.

4. The apparatus for fragrance sensory stimulation according to claim 1, wherein said plurality of fragrance chemicals are connected by a the micropump via a common conduit.

5. The apparatus for fragrance sensory stimulation according to claim 1, further comprising a fan.

6. The apparatus for fragrance sensory stimulation according to claim 1, further comprising a calibration system disposed adjacent the fragrance delivery system.

7. The apparatus for fragrance sensory stimulation according to claim 1, further comprising a microprocessor controller for controlling the sequence in which said fragrances are dispersed.

8. The apparatus for fragrance sensory stimulation according to claim 7, wherein the microprocessor controller is triggered by an activation signal.

9. The apparatus for fragrance sensory stimulation according to claim 7, wherein the microprocessor controller is an ASIC.

10. The apparatus for fragrance sensory stimulation according to claim 8 wherein a sequence of fragrances is dispersed after the activation signal is received.

11. The apparatus of claim 3, wherein the chemical includes one or more constituents that attract particulate matter when exposed to the dispersal needle.

12. A method of fragrance sensory stimulation comprising the steps of:
connecting a multimedia source comprising at least one audiovisual signal to an audiovisual display,
delivering fragrance information synchronized with the audiovisual signal;
spraying the substance using a micropump,
passing a discharge stream through an electrostatic spray device to create a delivery stream, and
controlling the micropump and spray device with an electronic device.

13. The method of claim 12, wherein the electronic device is one of a microprocessor or ASIC.

14. The method of claim 12, wherein the micropump is a piezoelectric pump and further comprising the step of removing particulates from the environment.
